(19) United States
(12) Patent Application Publication
(10) Pub. No.: US 2008/0009667 A1
(43) Pub. Date: Jan. 10, 2008

(54) METHODS AND APPARATUS FOR PROLAPSE REPAIR AND HYSTERECTOMY

(75) Inventors: Ross A. Longhini, Stillwater, MN (US); Matthew J. Olson, Crystal, MN (US); James E. Cox, Corcoran, MN (US); Richard C. Kaleta, Plymouth, MN (US); Kelly Ann Dockendorf, St. Paul, MN (US); John Jason Buysman, Minnetonka, MN (US); Jason Westrum Ogdahl, Robbinsdale, MN (US); John F. Otte, St. Anthony, MN (US); Jessica L. Roll, Minnetonka, MN (US)

Correspondence Address: OBLON, SPIVAK, MCCLELLAND MAIER & NEUSTADT, P.C., 1940 DUKE STREET, ALEXANDRIA, VA, 22314, US

(73) Assignee: AMS Research Corporation, Minnetonka, MN (US)

(21) Appl. No.: 11/760,190

(22) Filed: Jun. 8, 2007

Related U.S. Application Data
(60) Provisional application No. 60/811,776, filed on Jun. 8, 2006.

(51) Int. Cl. A61F 2/00

(52) U.S. Cl. 600/37

(57) ABSTRACT

A hybrid prolapse repair material comprising a polypropylene and a graft body attached together. Attachments are provided for detachably attaching a repair material to a needle. A needle and a system for using the needle are contemplated to get repair material closer to the ischial spine. Graft to arm attachment concepts are taught to couple a mesh to a graft body. Additionally, a hysterectomy tool is provided to allow a surgeon to track vital organs.

### CROSS REFERENCE TO RELATED APPLICATIONS

The present application claims the benefit of U.S. Provisional Application No. 60/811,776, filed Jun. 08, 2006 the entire disclosure of which is incorporated herein by reference.

### BACKGROUND OF THE INVENTION

1. Field of the Invention

This invention relates to urogenital surgery.

2. Description of the Related Art

Female genital prolapse has long plagued women. It is estimated by the U.S. National Center for Health Statistics that 247,000 operations for genital prolapse were performed in 1998. With the increasing age of the U.S. population, these problems will likely assume additional importance.

Vaginal prolapse develops when intra-abdominal pressure pushes the vagina outside the body. In a normal situation, the levator ani muscles close the pelvic floor. This results in little force being applied to the fascia and ligaments that support the genital organs. Increases in abdominal pressure, failure of the muscles to keep the pelvic floor closed, and damage to the ligaments and fascia all contribute to the development of prolapse. In addition, if a woman has a hysterectomy, the vaginal angle may be altered, causing increased pressure at a more acute angle, accelerating the prolapse.

There are generally two different types of tissue that make up the supportive structure of the vagina and uterus. First, there are fibrous connective tissues that attach these organs to the pelvic walls (cardinal and uterosacral ligaments; pubocervical and rectovaginal fascia). Second, the levator ani muscles close the pelvic floor so the organs can rest on the muscular shelf thereby provided. It is when damage to the muscles open the pelvic floor or during the trauma of childbirth that the fascia and ligaments are strained. Breaks in the fascia allow the wall of the vagina or cervix to prolapse downward.

Several factors have been implicated as being involved in genital prolapse in women. It is thought that individual women have differing inherent strength of the relevant connective tissue. Further, loss of connective tissue strength might be associated with damage at childbirth, deterioration with age, poor collagen repair mechanisms, and poor nutrition. Loss of muscle strength might be associated with neuromuscular damage during childbirth, neural damage from chronic straining, and metabolic diseases that affect muscle function. Other factors involved in prolapse include increased loads on the supportive system, as seen in prolonged lifting or chronic coughing from chronic pulmonary disease, or some disturbance in the balance of the structural support of the genital organs. Obesity, constipation, and a history of hysterectomy have also been implicated as possible factors.

The common clinical symptoms of vaginal prolapse are related to the fact that, following hysterectomy, the vagina is inappropriately serving the role of a structural layer between intra-abdominal pressure and atmospheric pressure. This pressure differential puts tension on the supporting structures of the vagina, causing a “dragging feeling” where the tissues connect to the pelvic wall or a sacral backache due to traction on the uterosacral ligaments. Exposure of the moist vaginal walls leads to a feeling of perineal wetness and can lead to ulceration of the exposed vaginal wall. Vaginal prolapse may also result in loss of urethral support due to displacement of the normal structural relationship, resulting in stress urinary incontinence. Certain disruptions of the normal structural relationships can result in urinary retention, as well. Stretching of the bladder base is associated with vaginal prolapse and can result in complaints of increased urinary urgency and frequency. Other symptoms, such as anal incontinence and related bowel symptoms, and sexual dysfunction are also frequently seen with vaginal prolapse.

Anterior vaginal wall prolapse causes the vaginal wall to fail to hold the bladder in place. This condition, in which the bladder sags or drops into the vagina, is termed a cystocele. There are two types of cystocele caused by anterior vaginal wall prolapse. Paravaginal defect is caused by weakness in the lateral supports (pubourethral ligaments and attachment of the bladder to the endopelvic fascia); central defect is caused by weakness in the central supports. There may also be a transverse defect, causing cystecele across the vagina.

Posterior vaginal wall prolapse results in descent of the rectum into the vagina, often termed a rectocele, or the presence of small intestine in a hernia sac between the rectum and vagina, called an enterocele. Broadly, there are four types based on suspected etiology. Congenital enteroceles are thought to occur because of failure of fusion or reopening of the fused peritoneal leaves down to the perineal body. Posthysterectomy vault prolapses may be “pulsion” types that are caused by pushing with increased intra-abdominal pressure. They may occur because of failure to reapproximate the superior aspects of the pubocervical fascia and the rectovaginal fascia at the time of surgery. Enteroceles that are associated with cystocele and rectocele may be from “traction” or pulling down of the vaginal vault by the prolapsing organs. Finally, iatrogenic prolapses may occur after a surgical procedure that changes the vaginal axis, such as certain surgical procedures for treatment of incontinence. With regard to rectoceles, low rectoceles may result from disruption of connective tissue supports in the distal posterior vaginal wall, perineal membrane, and perineal body. Mid-vaginal and high rectoceles may result from loss of lateral supports or defects in the rectovaginal septum. High rectoceles may result from loss of apical vaginal supports. Posterior or posthysterectomy enteroceles may accompany rectoceles.

As noted, vaginal prolapse and the concomitant anterior cystocele can lead to discomfort, urinary incontinence, and incomplete emptying of the bladder. Posterior vaginal prolapse may additionally cause defecatory problems, such as tenesmus and constipation.

Many techniques have been tried to correct or ameliorate the prolapse and its symptoms, with varying degrees of success. Nonsurgical treatment of prolapse involves measures to improve the factors associated with prolapse, including treating chronic cough, obesity, and constipation. Other nonsurgical treatments may include pelvic muscles exercises or supplementation with estrogen. These therapies may alleviate symptoms and prevent worsening, but the actual hernia will remain. Vaginal pessaries are the primary type of nonsurgical treatment, but there can be complications due to vaginal wall ulceration.

There are a variety of known surgical techniques for the treatment of anterior vaginal prolapses. In the small proportion of cases in which the prolapse is caused by a central defect, anterior colporrapphy is an option. This surgery involves a transvaginal approach in which plication sutures are used to reapproximate the attenuated tissue across the midline of the vagina. More commonly, the prolapse is due to a lateral defect or a combination of lateral and central defects. In these instances, several surgical techniques have been used, such as a combination of an anterior colporrapphy and a site-specific paravaginal repair. Both abdominal and vaginal approaches are utilized. Biological or synthetic grafts have been incorporated to augment repair.

One surgical technique for the repair of anterior vaginal prolapse can be accomplished with the Perigee™ System developed by American Medical Systems located in Minnetonka, Minn. In this transobturator anterior prolapse repair procedure, an incision is made medially along the anterior vaginal wall. The bladder is dissected off the vagina up to the lateral sulcus and posterior to the vaginal vault. Two small incisions are made over the obturator membrane along the pubic remus, one superior and one inferior. Curved needles are passed from skin incisions through the obturator foramen. The superior needles pass proximal to the vaginal vault and the inferior needles pass distal to the bladder neck. The needle tips are palpated via blunt dissection as they penetrate the obturator membrane. A graft is placed across the anterior vaginal wall. Connectors pre-attached to the sheaths enclosing the self-fixating mesh appendages are attached to the left and right superior needles and retracted. This is repeated for the inferior needles. Final adjustments are made and incisions closed.

Likewise, the treatment of posterior vaginal prolapses may vary. If symptoms are minimal, nonoperative therapy such as changes in activities, treatment of constipation, and Kegel exercises might be appropriate. Again, both vaginal and abdominal approaches are used, involving sutures to reapproximate the attenuated tissue and possibly a biological or synthetic graft to augment the repair.

A surgical technique for treating a vaginal vault prolapse can be accomplished with the Apogee™ vault suspension system developed by American Medical Systems located in Minnetonka, Minn. To use the Apogee™ system, an incision is made transversely across the vaginal apex to create access to the peritoneal cavity. Two small incisions are also made in the skin of the buttocks. Needles are passed from the skin incisions in the buttocks to the vaginal incision. The needle tip is palpated distal and inferior to the ischial spine prior to passage through the coccygeus muscle. Further dissection may be desired to aid palpation of the needle passage. Connectors are attached to each needle end. Needles are retracted and mesh is positioned. The mesh is then attached to the vaginal vault and the incisions are closed.

Examples of methods and apparatus useful for effecting repair of prolapse conditions include those disclosed in U.S. Publication 2005/0245787, herein expressly incorporated by reference, and in U.S. Publication 2005/0250977, also herein expressly incorporated by reference. U.S. Pat. No. 6,802,807, U.S. Pat. No. 6,911,003, U.S. Pat. No. 7,048,682, and U.S. Pat. No. 6,971,986 are also incorporated by reference.

Surgical techniques for prolapse repair often utilize surgical repair material such as a biological graft or a synthetic mesh. The biological graft can be made out of porcine dermis. A biological graft minimizes the risk of extrusions. The synthetic mesh can be made out of polypropylene. Polypropylene has desirable durability and improves usability. Clearly there is a need for a repair material which both combines the durability of polypropylene with the usability and decreased extrusion risk of biological grafts.

Repair material is attached to a needle for surgery. The attachment must be strong enough so that the repair material does not detach during implantation, but can still be detached from the needle when the surgeon desires. Variations in prolapse surgery techniques, the different pathology of patients, the preference of the surgeons, and the many kinds of needles and repair materials used during surgery create a need for many different types of connections between the needle and repair material.

In some prolapse surgery techniques it is desirable to attached the repair material to the ischial spine. Thus, it is desirable to have a system for getting the repair material to the ischial spine consistently, and a needle for implementing the system.

Repair material used for prolapse repair may comprise multiple articles of mesh that need to be attached together. It is desirable that the articles of mesh have a strong attachment, so that the repair material is durable. Thus, there is a need for types of attachment that are stronger than those currently in use.

During a hysterectomy, a surgeon may use multiple hemostats and sutures to remain oriented regarding the surgical anatomy, such as pertinent ligaments including the uterosacral and cardinal ligaments. The surgeon could greatly benefit from a tool which includes a built in retractor and suture attachment points for the ligaments.

### SUMMARY OF THE INVENTION

The present invention includes hybrid prolapse repair material, connections for attaching repair material to a needle, a system for getting the repair material closer to the ischial spine for attachment thereto, graft to arm attachment concepts, and a hysterectomy tool.

Many surgical techniques for repairing vaginal prolapse utilize a synthetic or biological graft. One embodiment of the present invention is a hybrid prolapse repair material comprised of polypropylene and porcine dermis. In a preferred embodiment, polypropylene can be connected to the porcine dermis at the arm attachment points and at each end.

In addition to polypropylene and porcine dermis, other suitable synthetic or non-synthetic material, or combinations thereof, are within the scope of the present invention. Suitable non-synthetic materials include allografts, homografts, heterografts, autologous tissues, cadaveric fascia, autodermal grafts, dermal collagen grafts, autofascial heterografts, whole skin grafts, porcine dermal collagen, lyophilized aortic homografts, preserved dural homografts, bovine pericardium and fascia lata. Commercial examples of synthetic materials include Marlex™ (polypropylene) available from Bard of Covington, R.I., Prolene™ (polypropylene), Prolene Soft Polypropylene Mesh or Gynemesh (nonabsorbable synthetic surgical mesh), both available from Ethicon, of New Jersey, and Mersilene (polyethylene terphthalate) Hernia Mesh also available from Ethicon, Gore-Tex.™ (expanded polytetrafluoroethylene) available from W. L. Gore and Associates, Phoenix, Ariz., and the polypropylene sling available in the SPARC™ sling system, available from American Medical Systems, Inc. of Minnetonka, Minn., Dexon™ (polyglycolic acid) available from Davis and Geck of Danbury, Conn., and Vicryl™ available from Ethicon.

Other examples of suitable materials include those disclosed in published U.S. patent application Ser. No. 2002/0072694, herein incorporated by reference. More specific examples of synthetic materials include, but are not limited to, polypropylene, cellulose, polyvinyl, silicone, polytetrafluoroethylene, polygalactin, Silastic, carbon-fiber, polyethylene, nylon, polyester (e.g. Dacron) polyanhydrides, polycaprolactone, polyglycolic acid, poly-L-lactic acid, poly-D-L-lactic acid and polyphosphate esters. See Cervigni et al., The Use of Synthetics in the Treatment of Pelvic Organ Prolapse, Current Opinion in Urology (2001), 11: 429-435.

Another aspect of the present invention is to provide novel structures to allow connections between the repair material and the needle for use in urogenital or other surgery which optimize manufacturability and ease of surgeon use. The repair material/needle attachment in this aspect of the present invention must be strong enough so that the attachment does not detach during implantation of the repair material, but the attachment must be sufficiently detachable to allow for relative ease in removing once the needle has to be retracted.

In one embodiment of the novel needle/repair material connection structure, a cap is placed over a tip of a needle to couple repair material to the tip of the needle.

In another embodiment of the novel needle/repair material connection structure, a needle may be hollow inside so repair material can be located in the hollow portion of the needle.

In another embodiment of the novel needle/repair material connection structure, a needle may have at least two eyelets and a hollow cap such that repair material can be threaded through the eyelets with an edge of the repair material located within the hollow cap.

In another embodiment of the novel needle/repair material connection structure, a retaining sleeve may be coupled to a needle such that repair material is attached to the needle via the retaining sleeve. Alternatively, the needle may contain an eyelet and two retaining sleeves may be coupled to one another, through the eyelet, such that the sleeves are also coupled to the needle. Repair material may be placed in between either sleeve, or between both sleeves, and the needle body such that the repair material is removably attached to the needle body when the two sleeves are coupled to one another.

In another embodiment of the novel needle/repair material connection structure, a hollow needle contains repair material within an inner opening of the hollow needle. The hollow needle also contains an opening in the exterior of the needle so the repair material may be removed from the needle through the exterior opening.

In another embodiment of the novel needle/repair material connection structure, repair material may be contained within a needle which can be opened on a hinge that is located co-axially to the needle.

In another embodiment of the novel needle/repair material connection structure, repair material may be coupled to a needle via a removable elastic sleeve.

In another embodiment of the novel needle/repair material connection structure, a needle tip may be removably attached to a body of the needle. Further, the repair material can be coupled to the needle when the needle tip is attached to the needle body.

In another embodiment of the novel needle/repair material connection structure, repair material can be located within a groove running the length of a needle with the repair material removably attached to the needle tip and to the needle handle.

In another embodiment of the novel needle/repair material connection structure, a suture is bonded to a tip of a needle and coupled to repair material.

In another embodiment of the novel needle/repair material connection structure, repair material may be coupled to a needle by wrapping the repair material and the needle in string or a strap.

In another embodiment of the novel needle/repair material connection structure, repair material may be coupled to the needle via a tightly fitting wrap.

In another embodiment of the novel needle/repair material connection structure, repair material is run through at least one eyelet in the needle and then heat sealed onto itself.

In another embodiment of the novel needle/repair material connection structure, a needle may contain an eyelet or a passage through the needle and repair material may contain a portion that is too large to be fed through the needle, such that the repair material will remain coupled to the needle until the large portion of the repair material is cut or the repair material is fed in reverse through the eyelet or passage.

In another embodiment of the novel needle/repair material connection structure, repair material may be formed such that the repair material may be coupled directly to the needle tip, or another protrusion along the needle.

In another embodiment of the novel needle/repair material connection structure, a needle may contain a flap that can be lifted up. Repair material may be placed under the flap and a ring, or another connector, may be coupled to the needle such that the flap is pinned to the needle body, trapping the repair material between the flap and the needle body.

Another aspect of the present invention is a system for consistently getting the repair material to the ischial spine in surgical procedures in which ischial attachment is required. A preferred embodiment is a novel specially shaped needle that allows consistent intraoperative placement of the repair material close to the ischial spine.

Another aspect of the present invention is to provide novel concepts for the attachment of arms of a mesh implant to a biological graft that provides increased attachment strength.

In an embodiment, a mesh arm is attached to a graft body with a suture and rivets with the mesh arm and the suture laying across the graft body, providing a seatbelt-like attachment.

In another embodiment, multiple mesh arms are attached to tabs of a graft body via a suture and rivets with the suture laid across the graft body, providing a seatbelt-like attachment.

In another embodiment, the size of a tab of a graft body is increased and a rivet is used to attach a mesh arm to the graft body.

In another embodiment, the size of a tab of a graft body is increased and a suture is used to attach a mesh arm to the graft body.

In another embodiment, the sizes of a tab of a graft body and an end of a mesh arm are increased and multiple rivets are used to attach the mesh arm to the graft body.

In another embodiment, the sizes of a tab of a graft body and an end of a mesh arm are increased and a large single rivet is used to attach the mesh arm to the graft body.

In another embodiment, the sizes of a tab of a graft body and an end of a mesh arm are increased and a suture is used to attach the mesh arm to the graft body.

In another embodiment, the sizes of a tab of a graft body and an end of a mesh arm are increased and the mesh arm and the graft body are melted together.

In another embodiment, the sizes of a tab of a graft body and an end of a mesh arm are increased and the end of the mesh arm is positioned to form a y-joint and attached to the graft body with a rivet.

In another embodiment, a graft body is shaped such that mesh arms are not used.

In another embodiment, tabs of a graft body are extended to form wings and mesh arms are attached to the wings with rivets.

In another embodiment, multiple arms are formed from a single piece of mesh and the mesh is attached to a graft body with rivets.

In another embodiment, multiple arms are formed from a single piece of mesh and the mesh is attached to a graft body with sutures.

Another aspect of the present invention is a hysterectomy tool.

In a preferred embodiment, a hysterectomy tool comprises a retractor portion and main body coupled to the retractor portion, with the main body having attachment points for sutures.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A depicts an embodiment of the hybrid prolapse repair material from a front view.

FIG. 1B depicts the same embodiment of the hybrid prolapse repair material depicted in FIG. 1A, from a rear view.

FIG. 2 depicts an embodiment of the needle/repair material attachment from a perspective view.

FIG. 3 depicts another embodiment of the needle/repair material attachment from a perspective view.

FIG. 4 depicts another embodiment of the needle/repair material attachment from a perspective view.

FIG. 5A depicts another embodiment of the needle/repair material attachment from a perspective view.

FIG. 5B depicts an alternate embodiment of the needle/repair material attachment to the embodiment shown in FIG. 5A.

FIG. 6 depicts another embodiment of the needle/repair material attachment from a perspective view.

FIG. 7A depicts another embodiment of the needle/repair material attachment from a sectional view.

FIG. 7B depicts the embodiment shown in FIG. 7A in an open position from a sectional view.

FIG. 8 depicts another embodiment of the needle/repair material attachment from a perspective view.

FIG. 9A depicts another embodiment of the needle/repair material attachment from a perspective view.

FIG. 9B depicts an alternate embodiment of the needle/repair material attachment as shown in FIG. 9A from a perspective view.

FIG. 10A depicts another embodiment of the needle/repair material attachment from a perspective view.

FIG. 10B depicts the needle/repair material attachment shown in FIG. 10A from a sectional view along line a′.

FIG. 11 depicts another embodiment of the needle/repair material attachment from a perspective view.

FIG. 12 depicts another embodiment of the needle/repair material attachment from a perspective view.

FIG. 13 depicts another embodiment of the needle/repair material attachment from a perspective view.

FIG. 14 depicts another embodiment of the needle/repair material attachment from a perspective view.

FIG. 15 depicts another embodiment of the needle/repair material attachment from a perspective view.

FIG. 16 depicts another embodiment of the needle/repair material attachment from a perspective view.

FIG. 17 depicts another embodiment of the needle/repair material attachment from a perspective view.

FIG. 18A depicts an embodiment of a needle for getting to the ischial spine from a side view.

FIG. 18B depicts the embodiment of the needle for getting to the ischial spine as depicted in 18A, from a top view.

FIG. 19 depicts an embodiment of the needle for getting to the ischial spine in use.

FIG. 20 depicts an embodiment of the needle for getting to the ischial spine in use.

FIG. 21 depicts an embodiment of the needle for getting to the ischial spine in use.

FIG. 22 depicts an embodiment of a graft to mesh arm attachment from a perspective view.

FIG. 23 depicts another embodiment of the graft to arm attachment from a perspective view.

FIG. 24 depicts another embodiment of the graft to arm attachment from a perspective view.

FIG. 25 depicts another embodiment of the graft to arm attachment from a perspective view.

FIG. 26 depicts another embodiment of the graft to arm attachment from a perspective view.

FIG. 27 depicts another embodiment of the graft to arm attachment from a perspective view.

FIG. 28 depicts another embodiment of the graft to arm attachment from a perspective view.

FIG. 29 depicts another embodiment of the graft to arm attachment from a perspective view.

FIG. 30 depicts another embodiment of the graft to arm attachment from a perspective view.

FIG. 31 depicts another embodiment of the graft to arm attachment from a perspective view.

FIG. 32 depicts another embodiment of the graft to arm attachment from a perspective view.

FIG. 33 depicts another embodiment of the graft to arm attachment from a perspective view.

FIG. 34 depicts an embodiment of the hysterectomy tool from a rear view.

FIG. 35 depicts the embodiment of the hysterectomy tool shown in FIG. 34, from a side view.

### DETAILED DESCRIPTION OF THE PREFERRED EMBODIMENTS

Referring now to the drawings, wherein like reference numerals designate identical or corresponding parts throughout the several views. The following description is meant to be illustrative only, and not limiting other embodiments of this invention will be apparent to those of ordinary skill in the art in view of this description.

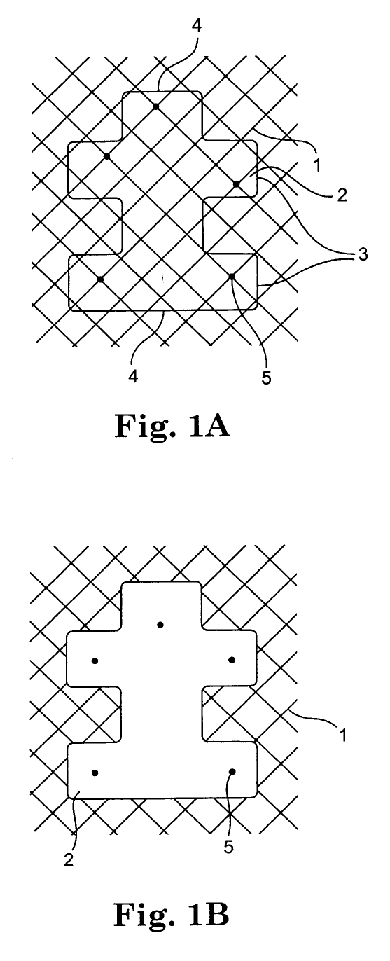

FIGS. 1A and 1B depict an embodiment of the hybrid prolapse repair material. As shown in FIG. 1A, a hybrid prolapse repair material is comprised of polypropylene 1 and a biological graft 2. The polypropylene can be connected to the biological graft at arm attachment points 3 and at each end 4. Rivets 5 may be used to connect the polypropylene to the biological graft. Other means to connect the polypropylene to the biological graft could include RivFix, suturing, melting, y-joints, or any other connection means commonly known in the art.

The polypropylene 1 and the biological graft 2 can be connected at the arm attachment points 3 and at an end 4, as shown in FIG. 1A. The connection means can be positioned elsewhere.

In use, the biological graft, as shown in FIG. 1B, would likely be placed on the vaginal side of the repair (anterior or posterior) to minimize the chance of extrusion.

An example of polypropylene used in the hybrid prolapse repair material could be Large Pore Polyproylene (LPP), or another suitable polypropylene commonly known in the art. An example of a biological graft could be InteXen LP, or another suitable biological graft.

The shape of the polypropylene and the biological graft shown in FIGS. 1A and 1B are for example purposes only, and are non-limiting. Alternate shapes of the polypropylene and the biological graft could be utilized as needed. Additionally, the hybrid prolapse repair material could include a biological graft on both sides of the polypropylene.

Another aspect of the present invention is to provide a connection between the repair material and the needle which optimizes manufacturability and ease of surgeon use. The attachment of the repair material to the needle for surgery must be strong enough so that the repair material does not detach during implantation. Additionally, the attachment must be sufficiently detachable to allow for relative ease in removing the repair material once the needle has to be retracted. Repair material may comprise a surgical mesh surrounded by a protective bag. Additionally, a leader may be attached to the protective bag or the surgical mesh. Alternative repair material may be utilized to correct a prolapse.

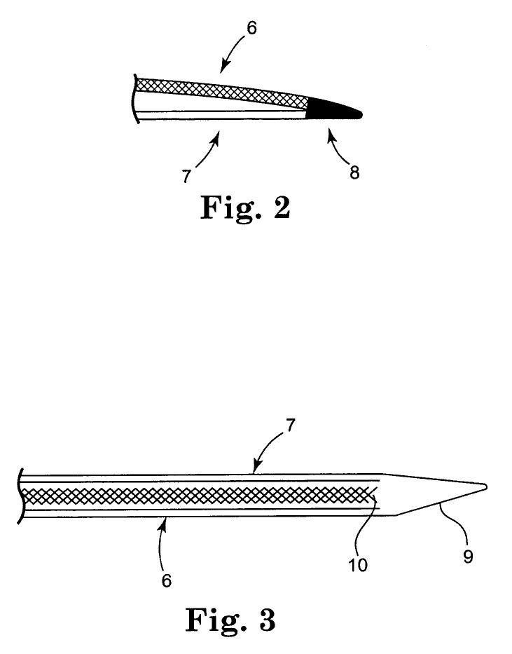

FIG. 2 depicts an illustrative embodiment of a connection between repair material 6 and a needle 7. The needle is coupled to the repair material via a cap 8 placed over the tip of the needle. Such a cap could have a cuff, or another type of protrusion, for easy detachment. Alternatively, the needle may have an eyelet to thread the repair material through for more effective coupling.

FIG. 3 depicts another illustrative embodiment. A needle 7 may be hollow inside. Repair material 6 can be located in the hollow portion 10 of the needle. The tip 9 of the needle may be broken off, or removed in another manner such as cutting, to allow detachment of the repair material. Alternatively, the tip of the needle may be opened up, such that it can be reattached to the body of the needle, for ease of extracting the needle.

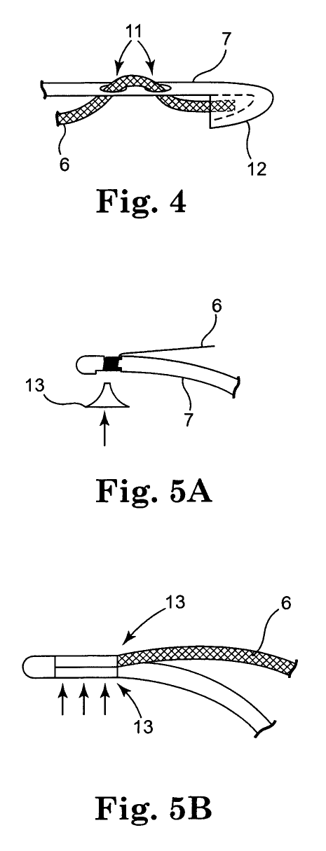

FIG. 4 depicts another illustrative embodiment. A needle 7 may have at least two eyelets 11 and a hollow cap 12. Repair material 6 can be threaded through the eyelets 11 such that an edge of the material may be located within the hollow cap 12. The repair material can be removed from the hollow cap for detachment.

FIGS. 5A and 5B depict another illustrative embodiment. As shown in FIG. 5A, a retaining sleeve 13 may be coupled to a needle 7. Repair material 6 is attached to the needle 7 via the retaining sleeve 13. When the retaining sleeve 13 is removed from the needle 7, the repair material 6 is detached. In an alternative embodiment, the needle 7 may contain two or more retaining sleeves 13, as shown in FIG. 5B. The two retaining sleeves 13 may be coupled to one another, through an eyelet (not shown), such that the sleeves are also coupled to the needle. Repair material 6 may be placed in between either of the sleeves, or between both of the sleeves, and the needle 7 body such that the repair material 6 is removably attached to the needle 7 body when the two sleeves are coupled to one another. To detach the repair material 6, one, or both of the sleeves 13, may be removed. The two sleeves may be coupled to each other by use of a male and female portion of each sleeve, they may be screwed together, they may snap together, or may be coupled by any means understood by one of ordinary skill in the art. Further, once the repair material is detached, the retaining sleeves may be reattached to the needle for ease of extraction.

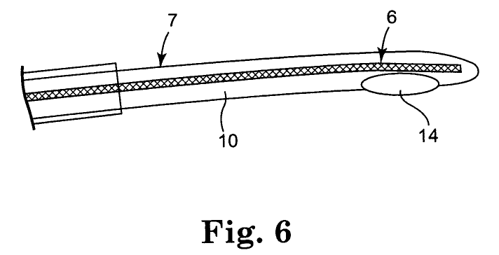

FIG. 6 depicts another illustrative embodiment. A hollow needle 7 contains repair material 6 within a hollow portion 10 of the hollow needle 7. The hollow needle 7 also contains an opening 14 in the exterior of the needle such that the repair material may be detached from the needle through the exterior opening.

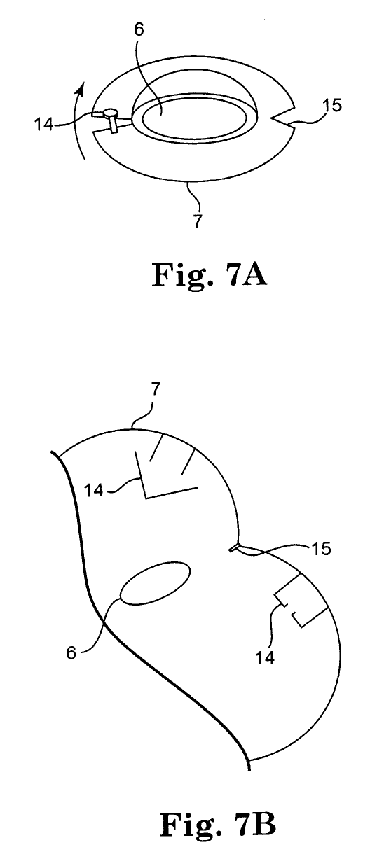

FIGS. 7A and 7B depict another illustrative embodiment. A needle 7 may contain repair material 6 within the needle. A latch 14 as shown in FIG. 7A, or equivalent fixing means, may be provided on the needle 7 such that the needle does not open during implantation or extraction. The needle can be opened on a hinge 15, or equivalent opening means, that is located co-axially to the needle such that the repair material may be removed as shown in FIG. 7B.

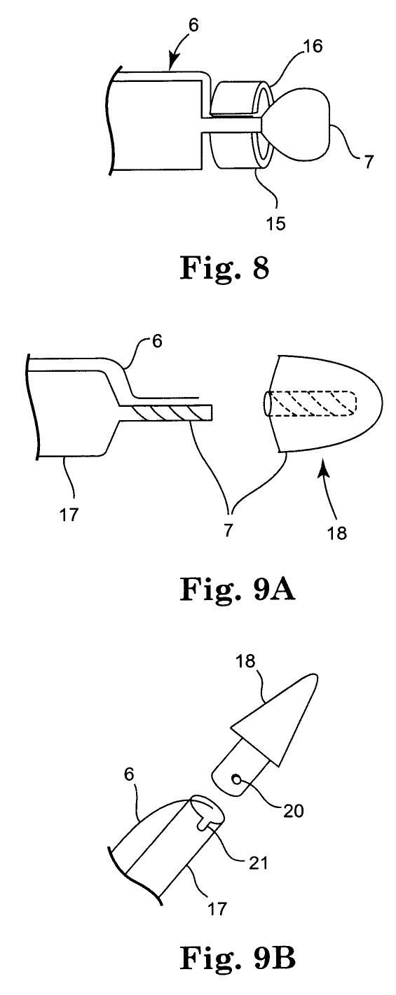

FIG. 8 depicts another illustrative embodiment. Repair material 6 may be coupled to a needle 7 via an elastic sleeve 15. The elastic sleeve 15 can be removed from the needle 7 so that the repair material 6 may be detached. The needle may have a notch 16 so that the elastic sleeve is securely fashioned to the needle for transporting the repair material during implantation.

FIGS. 9A and 9B depict illustrative embodiments. As shown in FIG. 9A, a needle tip 18 may be removably attached to a body 17 of a needle 7. Further, repair material 6 can be coupled to the needle 7 when the needle tip 18 is attached to the needle body 17. To detach the repair material 6, the needle tip is removed from the needle body. The needle tip may be screwed or snapped onto the needle 7 or the needle tip 18 may contain a pin 20 that locks the needle tip 18 into a slot 21 in the needle body 17 as shown in FIG. 9B, or any other method understood by one of ordinary skill in the art may be used.

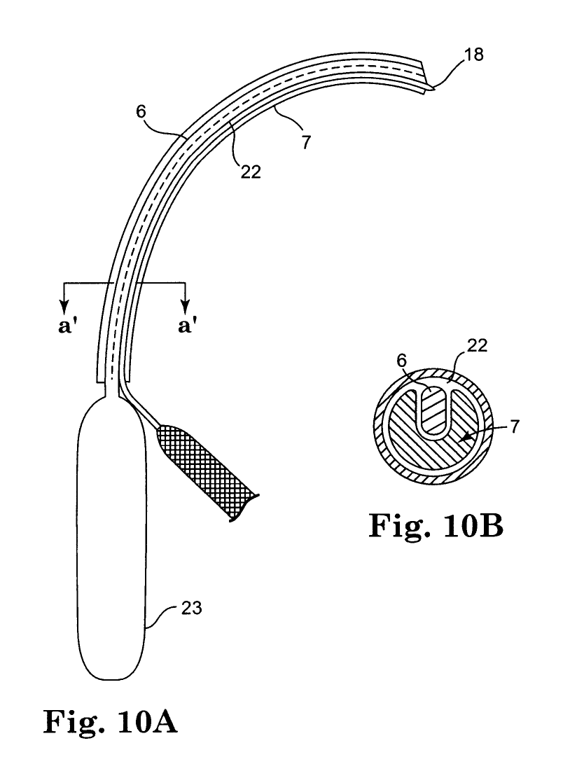

FIGS. 10A and 10B depict another illustrative embodiment. As shown in FIG. 10A, repair material 6 may be located within a groove 22 deposed lengthwise along a needle 7. The repair material 6 can be removably attached to the needle tip 18 and to the needle handle 23. Attachments could include sutures, melting, or any other attachment means. To detach the repair material, the means of attachment need to be removed. FIG. 10B shows the present embodiment along sectioned view a′ of FIG. 10A.

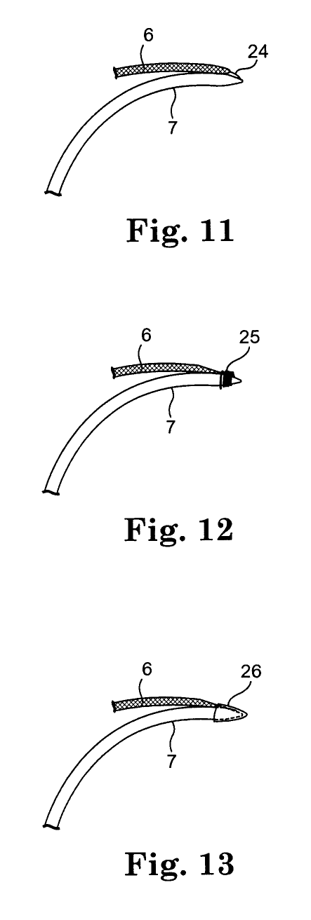

FIG. 11 depicts another illustrative embodiment. A suture 24 may be bonded to a tip of a needle 7. Further, the suture is coupled to repair material 6. To detach the repair material 6, the suture 24 may either be removed from the repair material, or the suture may be cut. Other attachments, such as melting, could be used to couple the repair material to the needle.

FIG. 12 depicts another illustrative embodiment. Repair material 6 may be coupled to a needle 7 by wrapping the repair material and the needle with string 25 or a strap. In order to detach the repair material, unwind or cut the string or strap.

FIG. 13 depicts another illustrative embodiment. Repair material 6 may be coupled to a needle 7 via a wrap 26 that fits tightly over a tip of the needle. The wrap 26 may be cut to detach the repair material 6. Alternatively, the wrap may contain a strip that, when pulled, detaches the repair material.

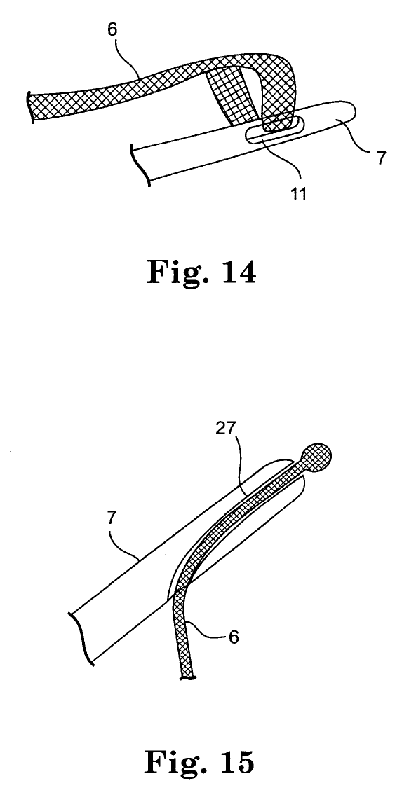

FIG. 14 depicts another illustrative embodiment. Repair material 6 may be run through at least one eyelet 11 in a needle 7. The repair material 6 can then be heat sealed onto itself such that it will not be detached from the needle 7 unless a portion of the repair material is cut. Alternatively, the repair material may be coupled to itself via suture, or any other means known to one of ordinary skill in the art.

FIG. 15 depicts another illustrative embodiment. A needle 7 may contain an eyelet or a passage 27 through the needle. Repair material may contain a portion of the repair material 6 that is too large to be fed through the needle 7. The repair material is partially fed through the eyelet or passage such that the repair material will remain coupled to the needle until the large portion of the repair material is cut or the repair material is fed in reverse through the eyelet or passage.

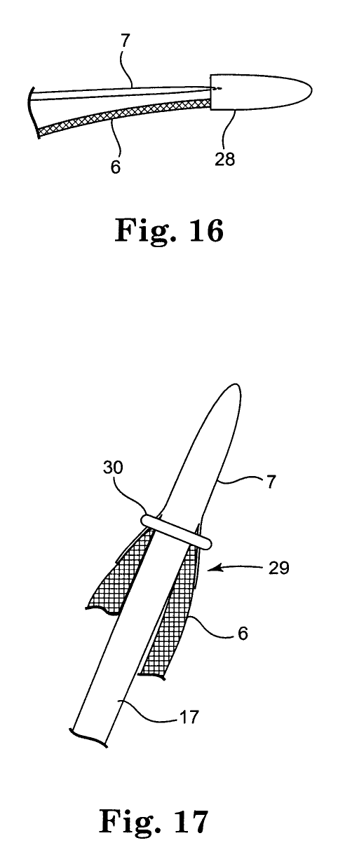

FIG. 16 depicts another illustrative embodiment. A portion of repair material 6 may be formed such that the repair material 6 can be coupled directly to a needle tip, or another protrusion along the needle 7. A portion of the repair material may be formed into a ring that can couple to the needle tip, or another protrusion along the needle. Alternatively, a portion of the repair material may be formed into a grommet 28, as shown, that couples to the needle tip, or another protrusion along the needle. The repair material may also be coupled to a ring, or a grommet, as opposed to forming the ring or grommet from the repair material. The repair material will remain coupled to the needle during implantation, but can be detached by simply repositioning the needle or the repair material so that the needle and the repair material are no longer coupled together.

FIG. 17 depicts another illustrative embodiment. A needle 7 may contain a flap 29 that can be lifted up. All or a portion of repair material 6 may be placed under the flap 29. A ring 30, or another connector, may be coupled to the needle 7 such that the flap 29 is pinned to the needle body 17, trapping the repair material 6 between the flap 29 and the needle body 17. The repair material 6 may be detached from the needle by removing the ring.

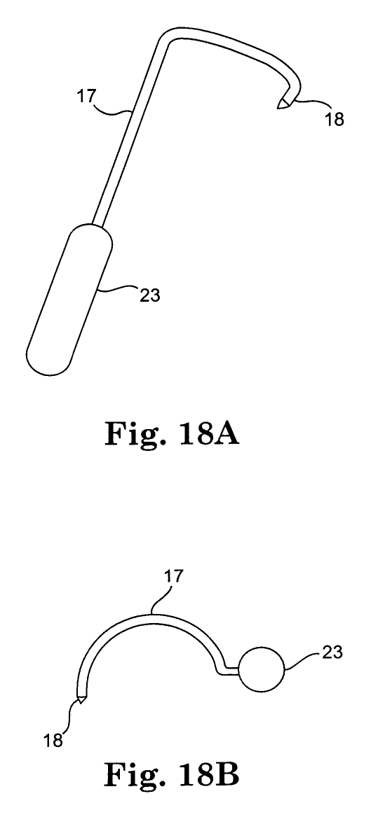

FIGS. 18A and 18B depict a needle for getting to the ischial spine. The needle comprises a handle 23, a needle body 17 and a needle tip 18. Often, a cannula 31 will be placed over top of the needle tip and body during surgery.

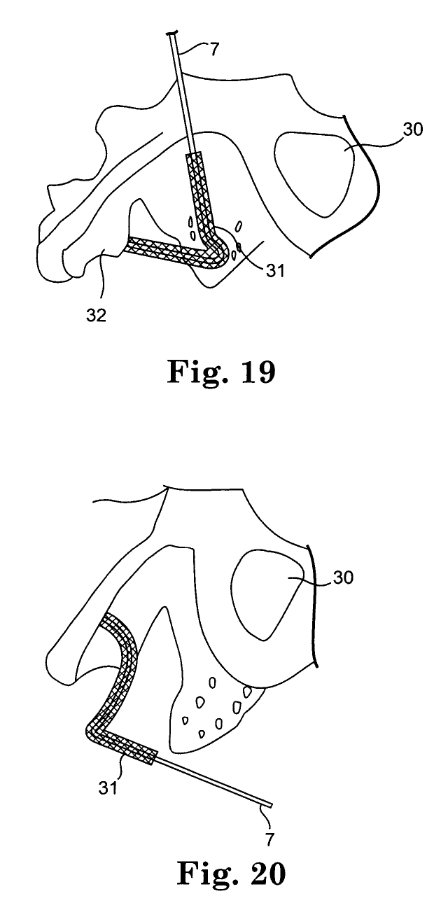

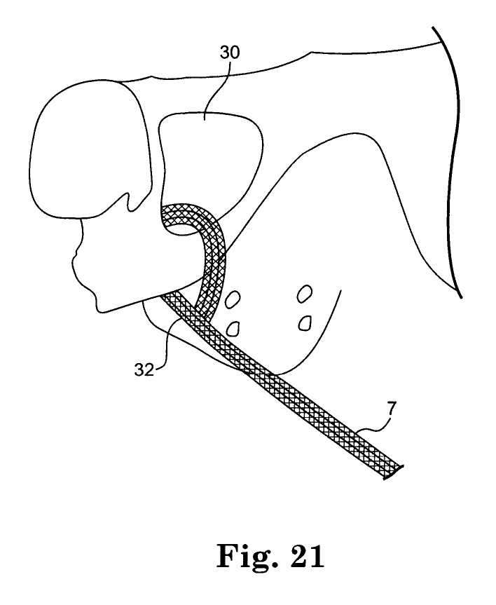

To utilize the needle, the needle tip 18 enters a vaginal incision and cuts through the levator ani muscles closest to the ischial spine 32 as shown in FIG. 19. Next, start rotating the needle such that the needle tip 18 moves along the pelvis as shown in FIG. 20. Continue rotating the needle 7 until it exits the body through the obturator foramen 30 as shown in FIG. 21. Next, remove the needle 7 leaving the cannula 31 behind. Run a dual lock through the cannula 31, attach it to repair material 6, pull the repair material through the cannula 31, and then remove the cannula 31.

In surgical procedures utilizing the Perigee™ or Apogee™ systems, multiple needles of the present embodiment could be used as both the superior and inferior needles.

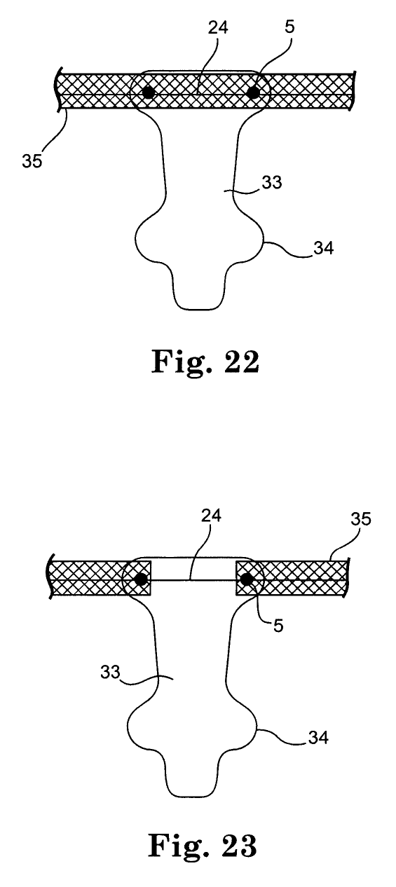

FIG. 22 depicts an illustrative embodiment of a mesh arm 35 attached to a graft body 33. The mesh arm 35 is attached to the graft body 33 with a suture 24 and rivets 5. The mesh arm 35 and the suture 24 lay across the graft body 33 like a seatbelt, and the mesh is attached by rivets 5 to tabs 34 of the graft body 33. The mesh may be SPARC, or another suitable surgical mesh. The graft body may be TiMesh, or another suitable surgical graft body. The suture may be a bioresorbable suture.

FIG. 23 depicts another illustrative embodiment. Multiple mesh arms 35 are attached to tabs 34 of a graft body 33 via a suture 24 and rivets 5. The rivets 5 are in tabs 34 of the graft body 33 and the suture 24 is laid across the graft body 33 like a seatbelt. One of ordinary skill in the art would understand that multiple sutures could be used and that the number of rivets used may vary.

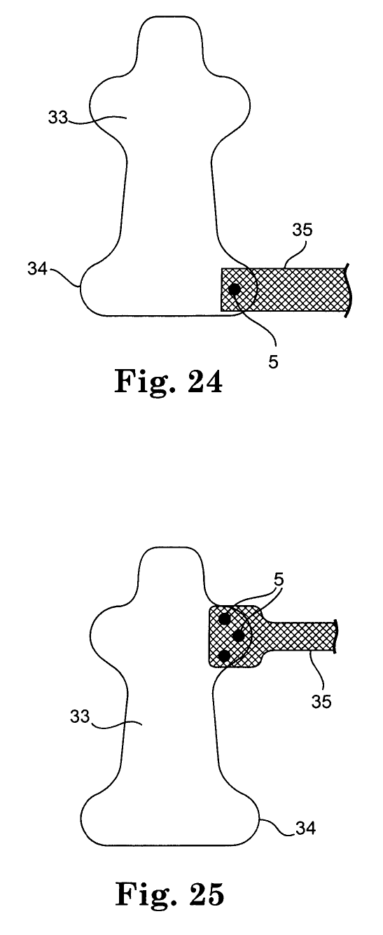

FIG. 24 depicts another illustrative embodiment. The size of a tab 34 of a graft body 33 is enlarged. A rivet 5 is used to attach a mesh arm 35 to the graft body 33.

FIG. 25 depicts another illustrative embodiment. The sizes of a tab 34 of a graft body 33 and an end of a mesh arm 35 are increased. Multiple rivets 5 are used to attach the mesh arm 35 to the graft body 33.

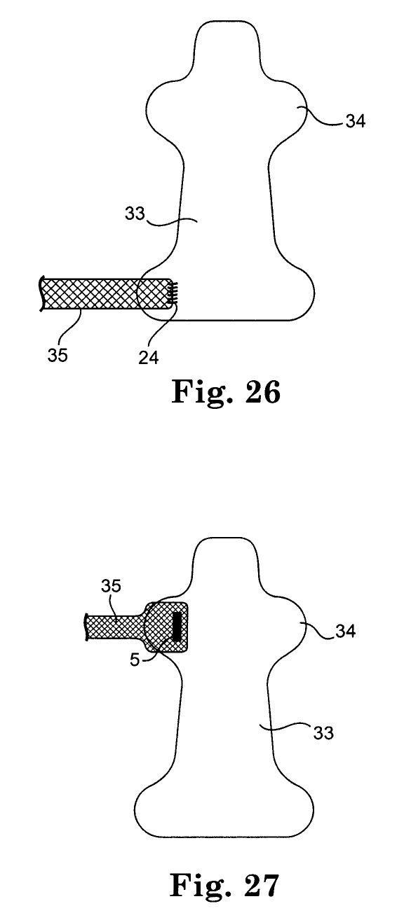

FIG. 26 depicts another illustrative embodiment. The size of a tab 34 of a graft body 33 is enlarged. A suture 24 is used to attach a mesh arm 35 to the graft body.

FIG. 27 depicts another illustrative embodiment. The sizes of a tab 34 of a graft body 33 and an end of a mesh arm 35 are increased. A large single rivet 5 is used to attach the mesh arm 35 to the graft body 33.

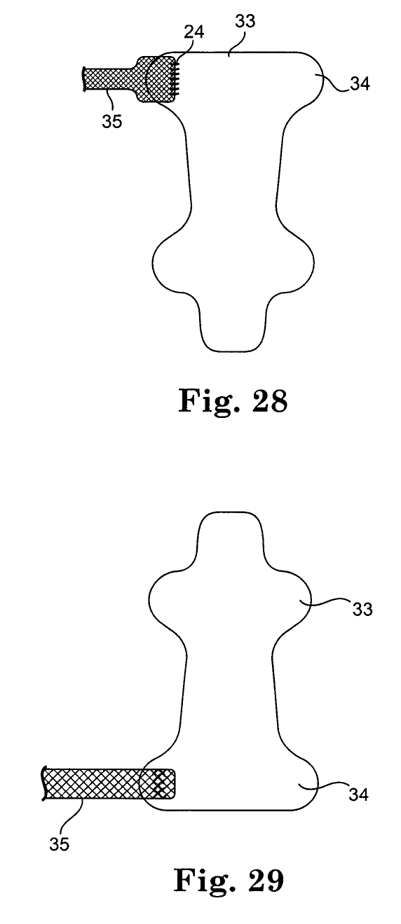

FIG. 28 depicts another illustrative embodiment. The sizes of a tab 34 of a graft body 33 and an end of a mesh arm 35 are increased. A suture 24 is used to attach the mesh arm 35 to the graft body 33.

FIG. 29 depicts another illustrative embodiment. The sizes of a tab 34 of a graft body 33 and an end of a mesh arm 35 are increased. The mesh arm 35 and the graft body 33 are melted together such that the mesh is attached to the graft body 33. The mesh arm 35 may be melted to the graft body 33 in a similar manner to the heat seal used on SPARC sheathes.

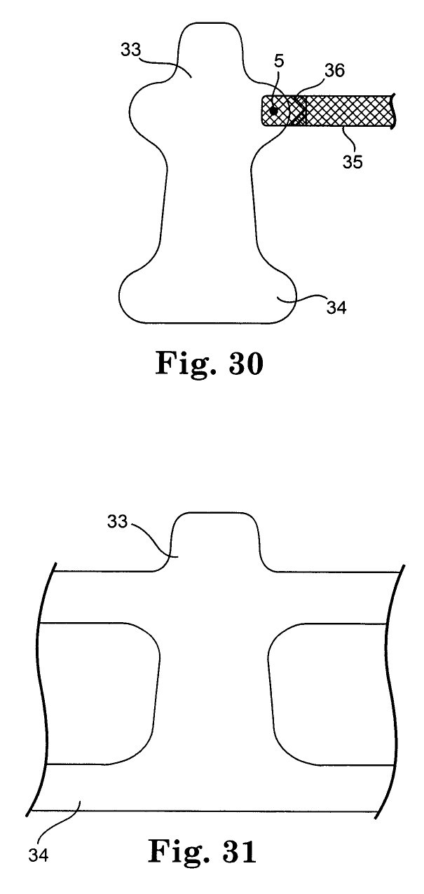

FIG. 30 depicts another illustrative embodiment. The sizes of a tab 34 of a graft body 33 and an end of a mesh arm 35 are increased. The end of the mesh arm 35 forms a y-joint 36 such that the mesh arm 35 contacts a front side and a back side of the graft body 33. The mesh arm 35 is attached to the graft body 33 with a rivet 5. The y-joint 36 may be similar to that utilized by InteXen LP in the Perigee™ system. Alternatively, the size of the mesh arm may not be increased, but still formed into a y-joint.

FIG. 31 depicts another illustrative embodiment. Tabs 34 on a graft body 33 are extended such that mesh arms are not used.

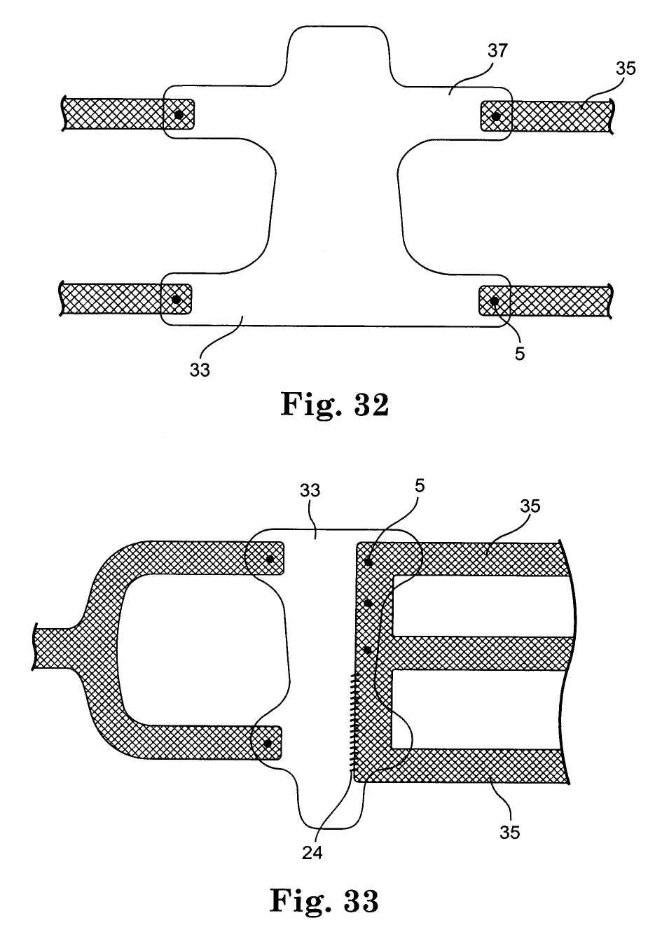

FIG. 32 depicts another illustrative embodiment. Tabs on a graft body 33 are extended to form wings 37 and mesh arms 35 are attached to the wings 37 with rivets 5 such that the attachment is further away from the main body. The mesh arms 35 may be a standard size, or the mesh arms may be enlarged.

FIG. 33 depicts another illustrative embodiment. Multiple arms 35 are formed from a single piece of mesh and the mesh is attached to a graft body 33 with rivets 5. The mesh may also be attached to the graft body with sutures 24.

The number and positioning of tabs on a graft body may be changed to meet different surgical requirements. A graft body may be provided without tabs, and the mesh may be attached to the graft body in a desired location. Additionally, different sizes and shapes of mesh may be utilized to meet different surgical requirements.

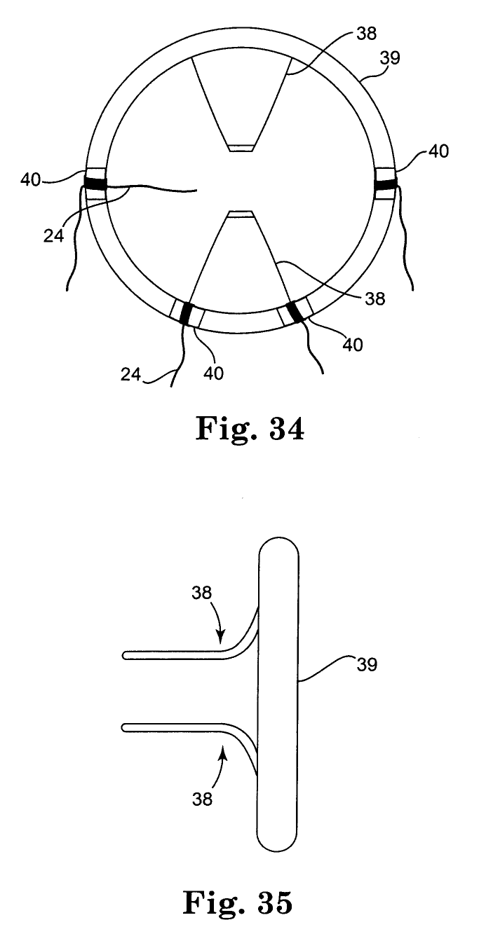

Another illustrative embodiment of the present invention is shown in FIGS. 34 and 35. As shown in FIG. 34, a hysterectomy tool includes a retractor portion 38. A main body 39 of the hysterectomy tool is coupled to the retractor portion 38. The main body 39 has attachment points 40 for sutures 24. The surgeon may tie sutures 24 to the attachment points 40, or the attachment points 40 may contain a device, such as a clasp, to secure the sutures. FIG. 35 shows the same embodiment of the hysterectomy tool from a side view.

In practice, a surgeon would use the hysterectomy tool by first inserting the retractor portion. Next, after the uterosacral and cardinal ligaments are tagged, the sutures are coupled to the attachment points on the main body of the hysterectomy tool. The sutures may be tied to the attachment points, or the attachment points may contain a means for holding the sutures such as a clamp, or a narrow groove.

Obviously, numerous modifications and variations of the present invention are possible in light of the above teachings. It is therefore to be understood that within the scope of the appended claims, the invention may be practiced otherwise than as specifically described herein.

1. A prolapse repair material comprising:
a biological component, and
a synthetic component,
wherein said biological component comprises at least one arm having at least one attachment point adapted for attaching to said biological component.

2. The material of claim 1, wherein said synthetic component comprises polypropylene.

3. The material of claim 1, wherein said biological component comprises porcine dermal material.

4. The material of claim 1, wherein said synthetic component and said biological component are attached together by at least one method of the group consisting of riveting, suturing, melting, and y-jointing.

5. The material of claim 1, said synthetic component having a front side and a back side, said material comprising a biological component attached to the front side of said synthetic component, and further comprising a biological component attached to the back side of said synthetic component.

6. A needle adapted for detachably coupling to a prolapse repair material.

7. The needle of claim 6, comprising a cap, said cap being tightly disposed to said needle such that the prolapse repair material is pinned between said cap and needle, wherein said cap is a dilator.

8. The needle of claim 7, wherein said cap comprises a protrusion adapted to facilitate removal of said cap.

9. The needle of claim 6, comprising:
a needle tip that is adapted to be opened or removed, and
a hollow portion adjacent to said tip adapted to hold said repair material prior to dispensing, wherein said prolapse repair material is dispensed upon opening or removing said needle tip.

10. The needle of claim 6, wherein said needle comprises at least two eyelets and a hollow cap, wherein said prolapse repair material is threaded through said eyelets such that an edge of the material is located in said cap, wherein said repair material is dispensed by removal from said cap.

11. The needle of claim 6 in combination with at least one retaining sleeve, said retaining sleeve detachably coupled to said needle, wherein said prolapse repair material is dispensed upon the uncoupling of said at least one retaining sleeve from said needle.

12. The needle of claim 6, comprising:
a hollow portion adapted to hold said prolapse repair material, and
a passageway between said hollow portion and the exterior of said needle,
wherein said prolapse repair material is dispensed through said opening.

13. The needle of claim 12, further comprising a latch and a hinge adapted to open and close said passageway.

14. The needle of claim 6, said needle comprising
a body, and
a needle tip, said needle tip removably attached to said body,
wherein said prolapse repair material is dispensed upon removal of said needle tip.

15. The needle of claim 14, further comprising threads or snaps adapted for attaching said needle tip to said body.

16. The needle of claim 14, wherein said needle body comprises a slot and said needle tip comprises a pin, said pin and slot adapted for attaching said needle tip to said body.

17. The needle of claim 6, further comprising a longitudinal groove adapted to hold said prolapse repair material.

18. A hysterectomy tool comprising:
a main body, and
a retractor portion disposed to the main body, wherein said retractor portion is adapted to retract the edges of an incision or other impeding structure and said main body comprises attachment points adapted for intraoperative suture and critical structure management.

19. A method for treating pelvic organ prolapse or incontinence by implanting a repair material, said method comprising the steps of locating a needle at the ischial spine, followed by rotating the needle until it exits through an obturator foramen.

20. The method of claim 19, wherein said needle is initially inserted in combination with a cannula, said method further comprising the steps of:
removing said needle following its exit through an obturator foramen, leaving said cannula in place, and
implanting said repair material through said cannula.
